SEQUENCE LISTING (28 sequences)

SEQ ID NO: 1 - DNA, 1098 bp, Solanum lycopersicum
  source 1..1098: mol_type = genomic DNA
atgactaatcagaatgtgataatttctgacccgagatcaggattcgactcttcactcttatcattgtcccctgccgttcccggtccactaccgcagccgggaagattcatcgctgttccagcgaagagatcattcaaaaatattgattctactgatgcagctagaatcactgctttacttgattccatgagagcttcttctccaactcgaagatcctcagaaactgaaaatctcaactcctggattgttcatcatccatcagctctgaatatgttcgatgaaattataaattcttcgaaaggaaaacaaattgtagtgtttttggattatgatggtacattgtctcctattgttgatgatcctgataaagccttcatgacttgtgagatgagggaagcagtaagaaacacatcaaagtattttccaacagcaatagtgagtggaagatgcagagcaaaagtgtttaattttgtgaaattgtcagaattatattatgctggaagtcatggaatggacattaaggcgcctgttaaagggcgtaattatagaaaaggaaataatcaaactgtactctgccaacctgcaagagaatttttacccatgataagtgaggtatataaatctttagtggagaaaacaaaatctataccaggagctaaagtggaaaacaacaaattttgtttatccgtacatttccgtcgtgttgaagaaaagagatggactgaactagctgagcaagtgaaatcagtgactaaggaatacccaacgcttcgattaactcaaggaagaaaggttctggagattcgtcctagtattaaatgggacaagggaaatgcactcgaatttttgttggaatcattaggctatgcaaactcaaatgatgttttgcctatttatattggcgatgatcgaactgatgaagatgctttcaaggttttgcgtgatagaggacaaggctttggaatattagtgtccaaatcacctaaagaaactaatgctacttattctttgcaagaaccattagaggttatgtactttttgaatcgtttggtggagtggaaacaatcgtcgttacaacaaaaatatcagaagaaataa

SEQ ID NO: 2 - protein, 365 aa, Solanum lycopersicum
MTNQNVIISDPRSGFDSSLLSLSPAVPGPLPQPGRFIAVPAKRSFKNIDSTDAARITALLDSMRASSPTRRSSETENLNSWIVHHPSALNMFDEIINSSKGKQIVVFLDYDGTLSPIVDDPDKAFMTCEMREAVRNTSKYFPTAIVSGRCRAKVFNFVKLSELYYAGSHGMDIKAPVKGRNYRKGNNQTVLCQPAREFLPMISEVYKSLVEKTKSIPGAKVENNKFCLSVHFRRVEEKRWTELAEQVKSVTKEYPTLRLTQGRKVLEIRPSIKWDKGNALEFLLESLGYANSNDVLPIYIGDDRTDEDAFKVLRDRGQGFGILVSKSPKETNATYSLQEPLEVMYFLNRLVEWKQSSLQQKYQKK

SEQ ID NO: 3 - DNA, 20 bp, Solanum lycopersicum
  source 1..20: mol_type = genomic DNA
ataatttctgacccgagatc

SEQ ID NO: 4 - DNA, 20 bp, Solanum lycopersicum
  source 1..20: mol_type = genomic DNA
tcccggtccactaccgcagc

SEQ ID NO: 5 - DNA, 1099 bp, synthetic construct
  source 1..1099: mol_type = other DNA
atgactaatcagaatgtgataatttctgacccgagaatcaggattcgactcttcactcttatcattgtcccctgccgttcccggtccactaccgcagccgggaagattcatcgctgttccagcgaagagatcattcaaaaatattgattctactgatgcagctagaatcactgctttacttgattccatgagagcttcttctccaactcgaagatcctcagaaactgaaaatctcaactcctggattgttcatcatccatcagctctgaatatgttcgatgaaattataaattcttcgaaaggaaaacaaattgtagtgtttttggattatgatggtacattgtctcctattgttgatgatcctgataaagccttcatgacttgtgagatgagggaagcagtaagaaacacatcaaagtattttccaacagcaatagtgagtggaagatgcagagcaaaagtgtttaattttgtgaaattgtcagaattatattatgctggaagtcatggaatggacattaaggcgcctgttaaagggcgtaattatagaaaaggaaataatcaaactgtactctgccaacctgcaagagaatttttacccatgataagtgaggtatataaatctttagtggagaaaacaaaatctataccaggagctaaagtggaaaacaacaaattttgtttatccgtacatttccgtcgtgttgaagaaaagagatggactgaactagctgagcaagtgaaatcagtgactaaggaatacccaacgcttcgattaactcaaggaagaaaggttctggagattcgtcctagtattaaatgggacaagggaaatgcactcgaatttttgttggaatcattaggctatgcaaactcaaatgatgttttgcctatttatattggcgatgatcgaactgatgaagatgctttcaaggttttgcgtgatagaggacaaggctttggaatattagtgtccaaatcacctaaagaaactaatgctacttattctttgcaagaaccattagaggttatgtactttttgaatcgtttggtggagtggaaacaatcgtcgttacaacaaaaatatcagaagaaataa

SEQ ID NO: 6 - DNA, 1099 bp, synthetic construct
  source 1..1099: mol_type = other DNA
atgactaatcagaatgtgataatttctgacccgagcatcaggattcgactcttcactcttatcattgtcccctgccgttcccggtccactaccgcagccgggaagattcatcgctgttccagcgaagagatcattcaaaaatattgattctactgatgcagctagaatcactgctttacttgattccatgagagcttcttctccaactcgaagatcctcagaaactgaaaatctcaactcctggattgttcatcatccatcagctctgaatatgttcgatgaaattataaattcttcgaaaggaaaacaaattgtagtgtttttggattatgatggtacattgtctcctattgttgatgatcctgataaagccttcatgacttgtgagatgagggaagcagtaagaaacacatcaaagtattttccaacagcaatagtgagtggaagatgcagagcaaaagtgtttaattttgtgaaattgtcagaattatattatgctggaagtcatggaatggacattaaggcgcctgttaaagggcgtaattatagaaaaggaaataatcaaactgtactctgccaacctgcaagagaatttttacccatgataagtgaggtatataaatctttagtggagaaaacaaaatctataccaggagctaaagtggaaaacaacaaattttgtttatccgtacatttccgtcgtgttgaagaaaagagatggactgaactagctgagcaagtgaaatcagtgactaaggaatacccaacgcttcgattaactcaaggaagaaaggttctggagattcgtcctagtattaaatgggacaagggaaatgcactcgaatttttgttggaatcattaggctatgcaaactcaaatgatgttttgcctatttatattggcgatgatcgaactgatgaagatgctttcaaggttttgcgtgatagaggacaaggctttggaatattagtgtccaaatcacctaaagaaactaatgctacttattctttgcaagaaccattagaggttatgtactttttgaatcgtttggtggagtggaaacaatcgtcgttacaacaaaaatatcagaagaaataa

SEQ ID NO: 7 - DNA, 1099 bp, synthetic construct
  source 1..1099: mol_type = other DNA
atgactaatcagaatgtgataatttctgacccgagatcaggattcgactcttcactcttatcattgtcccctgccgttcccggtccactaccgctagccgggaagattcatcgctgttccagcgaagagatcattcaaaaatattgattctactgatgcagctagaatcactgctttacttgattccatgagagcttcttctccaactcgaagatcctcagaaactgaaaatctcaactcctggattgttcatcatccatcagctctgaatatgttcgatgaaattataaattcttcgaaaggaaaacaaattgtagtgtttttggattatgatggtacattgtctcctattgttgatgatcctgataaagccttcatgacttgtgagatgagggaagcagtaagaaacacatcaaagtattttccaacagcaatagtgagtggaagatgcagagcaaaagtgtttaattttgtgaaattgtcagaattatattatgctggaagtcatggaatggacattaaggcgcctgttaaagggcgtaattatagaaaaggaaataatcaaactgtactctgccaacctgcaagagaatttttacccatgataagtgaggtatataaatctttagtggagaaaacaaaatctataccaggagctaaagtggaaaacaacaaattttgtttatccgtacatttccgtcgtgttgaagaaaagagatggactgaactagctgagcaagtgaaatcagtgactaaggaatacccaacgcttcgattaactcaaggaagaaaggttctggagattcgtcctagtattaaatgggacaagggaaatgcactcgaatttttgttggaatcattaggctatgcaaactcaaatgatgttttgcctatttatattggcgatgatcgaactgatgaagatgctttcaaggttttgcgtgatagaggacaaggctttggaatattagtgtccaaatcacctaaagaaactaatgctacttattctttgcaagaaccattagaggttatgtactttttgaatcgtttggtggagtggaaacaatcgtcgttacaacaaaaatatcagaagaaataa

SEQ ID NO: 8 - DNA, 1099 bp, synthetic construct
  source 1..1099: mol_type = other DNA
atgactaatcagaatgtgataatttctgacccgagatcaggattcgactcttcactcttatcattgtcccctgccgttcccggtccactaccgcaagccgggaagattcatcgctgttccagcgaagagatcattcaaaaatattgattctactgatgcagctagaatcactgctttacttgattccatgagagcttcttctccaactcgaagatcctcagaaactgaaaatctcaactcctggattgttcatcatccatcagctctgaatatgttcgatgaaattataaattcttcgaaaggaaaacaaattgtagtgtttttggattatgatggtacattgtctcctattgttgatgatcctgataaagccttcatgacttgtgagatgagggaagcagtaagaaacacatcaaagtattttccaacagcaatagtgagtggaagatgcagagcaaaagtgtttaattttgtgaaattgtcagaattatattatgctggaagtcatggaatggacattaaggcgcctgttaaagggcgtaattatagaaaaggaaataatcaaactgtactctgccaacctgcaagagaatttttacccatgataagtgaggtatataaatctttagtggagaaaacaaaatctataccaggagctaaagtggaaaacaacaaattttgtttatccgtacatttccgtcgtgttgaagaaaagagatggactgaactagctgagcaagtgaaatcagtgactaaggaatacccaacgcttcgattaactcaaggaagaaaggttctggagattcgtcctagtattaaatgggacaagggaaatgcactcgaatttttgttggaatcattaggctatgcaaactcaaatgatgttttgcctatttatattggcgatgatcgaactgatgaagatgctttcaaggttttgcgtgatagaggacaaggctttggaatattagtgtccaaatcacctaaagaaactaatgctacttattctttgcaagaaccattagaggttatgtactttttgaatcgtttggtggagtggaaacaatcgtcgttacaacaaaaatatcagaagaaataa

SEQ ID NO: 9 - DNA, 1099 bp, synthetic construct
  source 1..1099: mol_type = other DNA
atgactaatcagaatgtgataatttctgacccgagatcaggattcgactcttcactcttatcattgtcccctgccgttcccggtccactaccgccagccgggaagattcatcgctgttccagcgaagagatcattcaaaaatattgattctactgatgcagctagaatcactgctttacttgattccatgagagcttcttctccaactcgaagatcctcagaaactgaaaatctcaactcctggattgttcatcatccatcagctctgaatatgttcgatgaaattataaattcttcgaaaggaaaacaaattgtagtgtttttggattatgatggtacattgtctcctattgttgatgatcctgataaagccttcatgacttgtgagatgagggaagcagtaagaaacacatcaaagtattttccaacagcaatagtgagtggaagatgcagagcaaaagtgtttaattttgtgaaattgtcagaattatattatgctggaagtcatggaatggacattaaggcgcctgttaaagggcgtaattatagaaaaggaaataatcaaactgtactctgccaacctgcaagagaatttttacccatgataagtgaggtatataaatctttagtggagaaaacaaaatctataccaggagctaaagtggaaaacaacaaattttgtttatccgtacatttccgtcgtgttgaagaaaagagatggactgaactagctgagcaagtgaaatcagtgactaaggaatacccaacgcttcgattaactcaaggaagaaaggttctggagattcgtcctagtattaaatgggacaagggaaatgcactcgaatttttgttggaatcattaggctatgcaaactcaaatgatgttttgcctatttatattggcgatgatcgaactgatgaagatgctttcaaggttttgcgtgatagaggacaaggctttggaatattagtgtccaaatcacctaaagaaactaatgctacttattctttgcaagaaccattagaggttatgtactttttgaatcgtttggtggagtggaaacaatcgtcgttacaacaaaaatatcagaagaaataa

SEQ ID NO: 10 - DNA, 1097 bp, synthetic construct
  source 1..1097: mol_type = other DNA
atgactaatcagaatgtgataatttctgacccgagatcaggattcgactcttcactcttatcattgtcccctgccgttcccggtccactaccgagccgggaagattcatcgctgttccagcgaagagatcattcaaaaatattgattctactgatgcagctagaatcactgctttacttgattccatgagagcttcttctccaactcgaagatcctcagaaactgaaaatctcaactcctggattgttcatcatccatcagctctgaatatgttcgatgaaattataaattcttcgaaaggaaaacaaattgtagtgtttttggattatgatggtacattgtctcctattgttgatgatcctgataaagccttcatgacttgtgagatgagggaagcagtaagaaacacatcaaagtattttccaacagcaatagtgagtggaagatgcagagcaaaagtgtttaattttgtgaaattgtcagaattatattatgctggaagtcatggaatggacattaaggcgcctgttaaagggcgtaattatagaaaaggaaataatcaaactgtactctgccaacctgcaagagaatttttacccatgataagtgaggtatataaatctttagtggagaaaacaaaatctataccaggagctaaagtggaaaacaacaaattttgtttatccgtacatttccgtcgtgttgaagaaaagagatggactgaactagctgagcaagtgaaatcagtgactaaggaatacccaacgcttcgattaactcaaggaagaaaggttctggagattcgtcctagtattaaatgggacaagggaaatgcactcgaatttttgttggaatcattaggctatgcaaactcaaatgatgttttgcctatttatattggcgatgatcgaactgatgaagatgctttcaaggttttgcgtgatagaggacaaggctttggaatattagtgtccaaatcacctaaagaaactaatgctacttattctttgcaagaaccattagaggttatgtactttttgaatcgtttggtggagtggaaacaatcgtcgttacaacaaaaatatcagaagaaataa

SEQ ID NO: 11 - DNA, 1090 bp, synthetic construct
  source 1..1090: mol_type = other DNA
atgactaatcagaatgtgataatttctgacccgagtcaggattcgactcttcactcttatcattgtcccctgccgttcccggtccaagccgggaagattcatcgctgttccagcgaagagatcattcaaaaatattgattctactgatgcagctagaatcactgctttacttgattccatgagagcttcttctccaactcgaagatcctcagaaactgaaaatctcaactcctggattgttcatcatccatcagctctgaatatgttcgatgaaattataaattcttcgaaaggaaaacaaattgtagtgtttttggattatgatggtacattgtctcctattgttgatgatcctgataaagccttcatgacttgtgagatgagggaagcagtaagaaacacatcaaagtattttccaacagcaatagtgagtggaagatgcagagcaaaagtgtttaattttgtgaaattgtcagaattatattatgctggaagtcatggaatggacattaaggcgcctgttaaagggcgtaattatagaaaaggaaataatcaaactgtactctgccaacctgcaagagaatttttacccatgataagtgaggtatataaatctttagtggagaaaacaaaatctataccaggagctaaagtggaaaacaacaaattttgtttatccgtacatttccgtcgtgttgaagaaaagagatggactgaactagctgagcaagtgaaatcagtgactaaggaatacccaacgcttcgattaactcaaggaagaaaggttctggagattcgtcctagtattaaatgggacaagggaaatgcactcgaatttttgttggaatcattaggctatgcaaactcaaatgatgttttgcctatttatattggcgatgatcgaactgatgaagatgctttcaaggttttgcgtgatagaggacaaggctttggaatattagtgtccaaatcacctaaagaaactaatgctacttattctttgcaagaaccattagaggttatgtactttttgaatcgtttggtggagtggaaacaatcgtcgttacaacaaaaatatcagaagaaataa

SEQ ID NO: 12 - DNA, 309 bp, synthetic construct
  source 1..309: mol_type = other DNA
aggttttgcgtgatagaggacaaggctttggaatattagtgtccaaatcacctaaagaaactaatgctacttattctttgcaagaaccattagaggttatgtactttttgaatcgtttggtggagtggaaacaatcgtcgttacaacaaaaatatcagaagaaataaaagtactaaatctcagaagactttagctaaattaacacaaggaattgacccttttaaccctggaagttttattagggatggggtttaattaaaagaacttaatttgtatctttttcttcttggaagactcaactgtaattgg

SEQ ID NO: 13 - DNA, 21 bp, synthetic construct
  source 1..21: mol_type = other DNA
gagcacacattgagccttgac

SEQ ID NO: 14 - DNA, 20 bp, synthetic construct
  source 1..20: mol_type = other DNA
ctttggtacatcggcattcc

SEQ ID NO: 15 - DNA, 21 bp, synthetic construct
  source 1..21: mol_type = other DNA
aggttttgcgtgatagaggac

SEQ ID NO: 16 - DNA, 25 bp, synthetic construct
  source 1..25: mol_type = other DNA
ccaattacagttgagtcttccaaga

SEQ ID NO: 17 - DNA, 22 bp, synthetic construct
  source 1..22: mol_type = other DNA
attgataatttgacccgagatc

SEQ ID NO: 18 - DNA, 24 bp, synthetic construct
  source 1..24: mol_type = other DNA
aaacgatctcgggtcagaaattat

SEQ ID NO: 19 - DNA, 24 bp, synthetic construct
  source 1..24: mol_type = other DNA
attgtcccggtccactaccgcagc

SEQ ID NO: 20 - DNA, 24 bp, synthetic construct
  source 1..24: mol_type = other DNA
aaacgctgcggtagtggaccggga

SEQ ID NO: 21 - DNA, 24 bp, synthetic construct
  source 1..24: mol_type = other DNA
agtcacgacgttgtaaaacgacgg

SEQ ID NO: 22 - DNA, 20 bp, synthetic construct
  source 1..20: mol_type = other DNA
ctgcaggcatgcaagcttat

SEQ ID NO: 23 - DNA, 20 bp, synthetic construct
  source 1..20: mol_type = other DNA
atcgagcggaacataagtcc

SEQ ID NO: 24 - DNA, 18 bp, synthetic construct
  source 1..18: mol_type = other DNA
tgcaggttggcagtacag

SEQ ID NO: 25 - DNA, 19 bp, synthetic construct
  source 1..19: mol_type = other DNA
ttgcttggaggaacagacg

SEQ ID NO: 26 - DNA, 20 bp, synthetic construct
  source 1..20: mol_type = other DNA
gcaaacagaacccctgaatc

SEQ ID NO: 27 - DNA, 22 bp, synthetic construct
  source 1..22: mol_type = other DNA
ggttctggagattcgtcctagt

SEQ ID NO: 28 - DNA, 22 bp, synthetic construct
  source 1..22: mol_type = other DNA
ccaaagccttgtcctctatcac

(19) United States
(12) Patent Application Publication
(10) Pub. No.: US 2025/0361517 A1
(43) Pub. Date: Nov. 27, 2025

(54) GENE THAT CONTROLS PARTHENOCARPIC FRUIT FORMATION AND USES THEREOF

(71) Applicant: UNIVERSITY OF SEOUL INDUSTRY COOPERATION FOUNDATION, Seoul (KR)

(72) Inventors: Ji-Seong KIM, Seoul (KR); Jeongeun LEE, Seoul (KR)

(73) Assignee: UNIVERSITY OF SEOUL INDUSTRY COOPERATION FOUNDATION, Seoul (KR)

(21) Appl. No.: 19/214,669

(22) Filed: May 21, 2025

(30) Foreign Application Priority Data
May 23, 2024 (KR) 10-2024-0066926

(51) Int. Cl. C12N 15/82; C12N 15/11

(52) U.S. Cl. CPC C12N 15/8262; C12N 15/111; C12N 15/8218

(57) ABSTRACT

The present disclosure relates to a gene that controls parthenocarpy and uses thereof. Specifically, according to the disclosure, when the SlTPP4 gene in a plant is knocked down/knocked out, the plant may form parthenocarpic fruits. When the expression of the SlTPP4 gene g of the present disclosure is inhibited to induce parthenocarpy, the fruit formation rate is 90%, and the parthenocarpic plant exhibits facultative parthenocarpy, and thus may also form normal seeded fruits when pollination occurs. In addition, fruiting occurs well even in a high temperature stress environment.

### BACKGROUND

### 1. FIELD OF THE INVENTION

The present disclosure relates to a gene that controls parthenocarpic fruit formation and uses thereof.

### 2. BACKGROUND OF THE INVENTION

In general, in angiosperms, fruits can develop normally only after pollination and fertilization. However, in some angiosperms, a phenomenon occurs in which the ovary develops into a fruit without fertilization and the fruit is seedless. This phenomenon is called parthenocarpy.

Naturally, parthenocarpy occurs because, in bananas and tangerines, fertilization does not occur because the pollen does not develop, even though pollination occurs; in pineapples, fertilization does not occur due to self-incompatibility, even though pollination occurs; in pears and apples, the temperature is excessively low during the flowering period; and in persimmons, the plant is aged.

Although fruit development by pollination is greatly affected by the environment, fruit development by parthenocarpy has the advantage of being relatively less affected by the environment. For example, tomato mutants that form fruits even at high or low temperatures or at low light intensity have been reported. As another example, the fruit of parthenocarpic pepper has been reported to have a low susceptibility to blossom-end rot. In addition, due to their seedless characteristics, parthenocarpic crops such as parthenocarpic watermelon, grapes, and bananas have the advantage of being easier and more convenient to consume, which can increase marketability.

Due to these advantages, there are cases where parthenocarpy is artificially induced in the field of agriculture or horticulture. For example, there are known cases of crossbreeding pepper and ground cherry, which are distantly related species that cannot be fertilized, and methods of stabbing the ovary of tobacco with a platinum wire. More commonly, there are cases of treating the stigma of apple or watermelon with auxin, and cases of spraying gibberellin on grapes.

Auxin and gibberellin, plant hormones used to induce parthenocarpy, have been widely reported to be involved in fruit formation, although their exact roles are unknown. When either auxin or gibberellin acts on unfertilized ovules, fruit development occurs in many plant species. Because of their role in fruit setting, these hormones are also used in agriculture to improve fruit setting.

Under this background, the inventors of the present disclosure have identified a novel gene controlling parthenocarpy, Solyc04g072920, and developed, based on transformation, a plant capable of producing parthenocarpic fruits without separate mechanical or chemical treatment, and a method for producing the same.

### SUMMARY

Therefore, an object of the present disclosure is to provide a composition for inducing parthenocarpy in a plant, comprising, as an active ingredient, a SlTPP4 gene expression inhibitor, a SlTPP4 protein activity inhibitor, or a SlTPP4 gene mutagen, wherein the SlTPP4 protein comprises the amino acid sequence of SEQ ID NO: 2, and the SlTPP4 gene encodes the STPP4 protein.

Another object of the present disclosure is to provide a method for inducing parthenocarpy in a plant, comprising a step of inducing a loss-of-function mutation in a gene encoding a SlTPP4 protein comprising the amino acid sequence of SEQ ID NO: 2, inhibiting the expression of the gene, or inhibiting the activity of the SlTPP4 protein.

Still another object of the present disclosure is to provide a transgenic plant or plant part exhibiting a parthenocarpic phenotype by the method for inducing parthenocarpy in a plant.

The present disclosure relates to a gene that controls parthenocarpic fruit formation and uses thereof.

Hereinafter, the present disclosure is described in more detail.

One aspect of the present disclosure is a composition for inducing parthenocarpy in a plant, comprising, as an active ingredient, a SlTPP4 gene expression inhibitor, a SlTPP4 protein activity inhibitor, or a SlTPP4 gene mutagen, wherein the SlTPP4 protein comprises the amino acid sequence of SEQ ID NO: 2, and the SlTPP4 gene encodes the SlTPP4 protein. The SlTPP4 gene may comprise the nucleotide sequence of SEQ ID NO: 1, without being limited thereto.

As used herein, the term “parthenocarpy” refers to a phenomenon in which an ovary develops into a fruit without fertilization and the fruit is seedless. When the composition of the present disclosure is used to induce a loss-of-function mutation in the SlTPP4 gene of a plant or inhibit the expression of the gene, facultative parthenocarpy is induced. “Induction of facultative parthenocarpy” means that whether or not parthenocarpic fruit is formed varies depending on whether or not pollination occurs. That is, the term means that seedless parthenocarpic fruits are produced when no pollination occurs, and normal seeded fruits are produced when pollination occurs.

The nucleotide sequence of SEQ ID NO: 1 is the SlTPP4 gene located on chromosome 4 of tomato (Solanum lycopersicum), which encodes trehalose-6-phosphate phosphatase that is 365aa in length. The SlTPP4 gene was after solyc04g072920, which was confirmed to be one of the eight SlTPPs of tomato, and has been reported to be mainly expressed in the roots.

The present inventors have found that, when interference or loss-of-function mutation of the SlTPP4 gene in a tomato plant is induced, the tomato plant exhibits parthenocarpic characteristics, which means that seedless fruits develop when the flowers are not pollinated. On the other hand, even loss-of-function mutants of the SlTPP4 gene can develop normal seeded fruits when flowers are pollinated. This type of parthenocarpy is called facultative parthenocarpy because it appears only in the absence of pollination. Therefore, the SlTPP4 gene is responsible for the parthenocarpic phenotype in plants such as tomato, and enables control of parthenocarpy. The SlTPP4 loss-of-function mutant in the example of the present disclosure produces seedless parthenocarpic fruits from unpollinated flowers, and produces normal seeded fruits from pollinated flowers.

The SlTPP4 gene expression inhibitor may include, in the broadest sense, all substances that can inhibit SlTPP4 gene expression by acting on one or more steps of the SlTPP4 protein expression process. Specifically, the SlTPP4 gene expression inhibitor may be selected from the group consisting of an antisense nucleotide, small interfering RNA (siRNA), short hairpin RNA (shRNA), micro-RNA (miRNA), ribozyme, DNAyzme, and peptide nucleic acids (PNAs), which bind complementarily to a sequence of the SlTPP4 gene, a sequence complementary to the sequence of the SlTPP4 gene, or a fragment of the mRNA sequence of the SlTPP4 gene, thereby inhibiting expression of the SlTPP4 gene, without being limited thereto.

The antisense nucleotide binds (hybridizes) to a complementary nucleotide sequence of DNA, immature mRNA. or mature mRNA, as defined by Watson-Crick base pairing, thereby interrupting the flow of genetic information from DNA to protein.

The siRNA may be composed of a sense sequence selected from the mRNA nucleotide sequence of the gene encoding the SlTPP4 protein, and an antisense sequence complementary to the sense sequence. The sense sequence may be 15 to 30 mer in length, without being limited thereto.

The SlTPP4 protein activity inhibitor may be selected from the group consisting of a compound, a peptide, a peptide mimetic, a substrate analog, an aptamer, and an antibody, which bind to the SlTPP4 protein, without being limited thereto.

The compound includes any compound that may bind specifically to the SlTPP4 protein and inhibit the activity thereof.

The aptamer is a single-stranded DNA or RNA molecule, and may be obtained by isolating an oligomer, which binds to a specific chemical molecule or biological molecule with high affinity and selectivity, by an evolutionary method using an oligonucleotide library called SELEX (systematic evolution of ligands by exponential enrichment). The aptamer may bind specifically to a target and regulate the activity of the target. For example, the aptamer may block the function of the target by binding to the target

The SlTPP4 gene mutagen may be either a complex (ribonucleoprotein) of a guide RNA specific to the target nucleotide sequence of the SlTPP4 gene and an endonuclease protein, or a recombinant vector comprising a DNA encoding a guide RNA specific to the target nucleotide sequence of the SlTPP4 gene and a nucleic acid sequence encoding an endonuclease protein.

As used herein, the term “target gene” means a portion of DNA within the genome of a plant in which a mutation is to be induced through the present disclosure. The type of target gene is not limited, and the target gene may include both a coding region and a non-coding region. A person skilled in the art may select the target gene according to the desired mutation for the plant to be produced, depending on the purpose.

As used herein, the term “guide RNA” refers to RNA specific for DNA encoding a nucleotide sequence of a target gene, and refers to ribonucleic acid that complementarily binds to all or part of a target DNA nucleotide sequence and directs an endonuclease protein to the target DNA nucleotide sequence. The guide RNA refers to a dual RNA comprising two RNAS, namely, crRNA (CRISPR RNA) and tracrRNA (trans-activating crRNA) as components, or refers to a single-stranded guide RNA (sgRNA) comprising a first region including a sequence that is completely or partially complementary to a nucleotide sequence in the target gene and a second region including a sequence that interacts with an RNA-guided nuclease. However, as long as the RNA-guided nuclease is in a form that can be active in the target nucleotide sequence, any guide RNA may be included in the scope of the present disclosure without limitation, and may be appropriately selected according to techniques known in the art in consideration of the type of endonuclease used therewith or the microorganism from which the endonuclease is derived.

In addition, the guide RNA may be, but is not limited to, a guide RNA transcribed from a plasmid template, a guide RNA transcribed in vitro (e.g., an oligonucleotide double-strand), or a synthetic guide RNA.

In the composition for inducing parthenocarpy in a plant according to the present disclosure, the guide RNA is designed to be specific to the target nucleotide sequence of the SlTPP4 gene. For example, the target nucleotide sequence of the SlTPP4 gene may be composed of the nucleotide sequence of SEQ ID NO: 3 or SEQ ID NO: 4, without being limited thereto. The nucleotide sequence of SEQ ID NO: 3 or SEQ ID NO: 4 is the sequence of the first or second region where the PAM sequence (NGG) is located within the ORF of the SlTPP4 gene consisting of the nucleotide sequence of SEQ ID NO: 1.

In the composition for inducing parthenocarpy in a plant according to the present disclosure, the endonuclease protein may be at least one selected from the group consisting of Cas9, Cpf1 (CRISPR from Prevotella and

Francisella 1), TALEN (transcription activator-like effector nuclease), ZEN (zinc finger nuclease), or functional analogs thereof, and preferably may be a Cas9 protein, without being limited thereto.

In addition, the Cas9 protein may be at least one selected from the group consisting of a Cas9 protein derived from Streptococcus pyogenes, a Cas9 protein derived from Campylobacter jejuni, a Cas9 protein derived from Streptococcus thermophilus or Streptococcus aureus, a Cas9 protein derived from Neisseria meningitidis, a Cas9 protein derived from Pasteurella multocida, a Cas9 protein derived from Francisella novicida, and the like, without being limited thereto. The Cas9 protein or its genetic information thereof may be obtained from known databases such as GenBank of NCBI (National Center for Biotechnology Information).

The Cas9 protein is an RNA-guided DNA endonuclease enzyme that induces double-stranded DNA breaks. In order for the Cas9 protein to bind precisely to the target nucleotide sequence and cut the DNA strand, a short sequence of three nucleotides known as a Protospacer-Adjacent Motif (PAM) should be present next to the target nucleotide sequence, and the Cas9 protein forms a cleavage between the 3^{rd} and 4^{th} base pairs from the PAM sequence (NGG).

In the composition for inducing parthenocarpy in a plant according to the present disclosure, the guide RNA and the endonuclease protein may form a ribonucleoprotein complex and may function as an RNA-guided engineered nuclease (RGEN).

Another object of the present disclosure is a method for inducing parthenocarpy in a plant, comprising a step of inducing a loss-of-function mutation in a gene encoding a SlTPP4 protein comprising the amino acid sequence of SEQ ID NO: 2, or inhibiting the expression of the gene, or inhibiting the activity of the SlTPP4 protein.

The inducing of the loss-of-function mutation may be achieved by introducing into a cell of the plant either a complex (ribonucleoprotein) of a guide RNA specific to a target nucleotide sequence of the SlTPP4 gene and an endonuclease protein, or a recombinant vector comprising a DNA encoding a guide RNA specific to a target nucleotide sequence of the SlTPP4 gene and a nucleic acid sequence encoding an endonuclease protein, without being limited thereto.

The guide RNA specific to the target nucleotide sequence of the SlTPP4 gene and the endonuclease protein are as described above.

The CRISPR/Cas9 system used in the present disclosure is a gene editing method using the non-homologous end joining (NHEJ) mechanism that introduces a double-strand break at a specific location of a specific gene to be edited, thereby inducing an insertion-deletion (InDel) mutation by incomplete repair induced during the DNA repair process.

In the method for inducing parthenocarpy in a plant according to the present disclosure, the method for transducing the complex of the guide RNA and the endonuclease protein into plant cells may be suitably selected from a calcium/polyethylene glycol method for protoplasts, electroporation of protoplasts, microinjection into plant material, (DNA or RNA-coated) particle bombardment of various plant materials, infection with (non-integrative) viruses in Agrobacterium tumefaciens mediated gene transfer, and the like.

In addition, introducing into a cell of the plant the recombinant vector comprising the DNA encoding the guide RNA specific to the target nucleotide sequence and the nucleic acid sequence encoding the endonuclease protein is referred to as a transformation method. Transformation of plant species is now routine for plant species, including both the Dicotyledoneae as well as the Monocotyledoneae. In principle, any transformation method may be used to introduce a recombinant vector according to the present disclosure into a suitable ancestor cell.

The “plant cell” into which the guide RNA specific to the target nucleotide sequence and the endonuclease protein are to be introduced may be any plant cell. The plant cell is a cultured cell, a cultured tissue, a cultured organ, or a whole plant. “Plant tissue” includes differentiated or undifferentiated plant tissues, for example, but not limited to, root, stem, leaf, pollen, seed, cancerous tissue, and various types of cells that are used for culture, i.e., single cell, protoplast, shoot, and callus tissue. The plant tissue may be in planta or in organ culture, tissue culture, or cell culture.

In the method for inducing parthenocarpy in a plant according to the present disclosure, any method known in the art may be used to regenerate the plant with the loss-of-function mutation induced. Genome-edited plant cells should be regenerated into a whole plant. Techniques for the regeneration of mature plants from callus or protoplast culture are well known in the art for a large number of different species.

The inhibition of expression of the gene may be achieved by treatment with an SlTPP4 gene expression inhibitor selected from the group consisting of an antisense nucleotide, small interfering RNA (siRNA), short hairpin RNA (shRNA), micro-RNA (miRNA), ribozyme, DNAyzme, and peptide nucleic acids (PNAs), which bind complementarily to the mRNA sequence of the SlTPP4 gene, a sequence complementary to the mRNA sequence of the SlTPP4 gene, or a fragment of the mRNA sequence of the SlTPP4 gene, thereby inhibiting the expression of the SlTPP4 gene, without being limited thereto.

Another aspect of the present disclosure is a transgenic plant or plant part exhibiting a parthenocarpic phenotype by the method for inducing parthenocarpy in a plant.

In a method according to one embodiment of the present disclosure, the plant may be a food crop selected from the group consisting of rice, wheat, barley, corn, soybean, potato, wheat, red bean, oat, and sorghum; a vegetable crop selected from the group consisting of Arabidopsis, Chinese cabbage, radish, pepper, strawberry, tomato, watermelon, cucumber, cabbage, melon, pumpkin, green onion, onion, and carrot; a special crop selected from the group consisting of ginseng, tobacco, cotton, sesame, sugarcane, sugar beet, perilla, peanut, and rapeseed; a fruit tree selected from the group consisting of apple trees, pear trees, jujube trees, peaches, kiwis, grapes, citrus fruits, persimmons, plums, apricots, and bananas; a flowering plant selected from the group consisting of roses, gladiolus, gerberas, carnations, chrysanthemums, lilies, and tulips; or a forage crop selected from the group consisting of ryegrass, red clover, orchardgrass, alfalfa, tall fescue, and perennial ryegrass.

Preferably, the plant may be a dicotyledonous plant such as tomato, Arabidopsis, potato, eggplant, tobacco, pepper, burdock, crown daisy, lettuce, bellflower, spinach, chard, sweet potato, celery, carrot, water parsley, parsley, Chinese cabbage, cabbage, mustard greens, watermelon, melon, cucumber, pumpkin, gourd, strawberry, soybean, mung bean, kidney bean, or pea. More preferably, the plant is tomato.

The plant part may be a fruit, a flower, a leaf, a stem, a cutting, an ovule, pollen, a root, a seed, a rhizome, a scion, an embryo, anther, a cell, or a protoplast.

The plant may contain a mutant of SlTPP4 gene represented by a nucleotide sequence selected from the group consisting of SEQ ID NO: 5 to SEQ ID NO: 11, without being limited thereto.

The present disclosure relates to a gene that controls parthenocarpy and uses thereof. Specifically, when the expression of the gene of the present disclosure is inhibited or a loss-of-function mutation in the gene is induced, it is possible to produce a plant capable of forming parthenocarpic fruits. The parthenocarpic plant produced by the present disclosure exhibits facultative parthenocarpy, and thus may form seedless parthenocarpic fruits when pollination does not occur, and may produce seeded fruits, like those in general plants, when pollination occurs.

The parthenocarpic fruit of the present disclosure exhibits a fruit formation rate of up to 90% of that of a wild-type pollinated fruit, and also a fresh weight of 70% of that of the wild-type pollinated fruit. In addition, it was confirmed that it is possible to form parthenocarpic fruits even under high-temperature stress conditions in which a wild-type plant cannot form pollinated fruits.

### BRIEF DESCRIPTION OF THE DRAWINGS

FIG. 1 relates to phylogenetic and expression analysis of Solyc04g072920. Specifically, (A) shows a phylogenetic tree of Solyc04g072920 (arrow) and orthologous genes, and (B) shows the expression level of Solyc04g072920 in various tissues from the wild type.

FIG. 2 relates to parthenocarpic fruit formation of Solyc04g072920 RNA interference lines (RNAi). Specifically, (A) shows the expression level of Solyc04g072920, (B) shows parthenocarpic fruits (bar=1 cm), and (C) shows the percentage of parthenocarpic fruit formation (N≥34). Wild type (WT) and Solyc04g07292-RNAi lines (D1, D3 and D8).

FIG. 3 relates to the CRISPR/Cas9-mediated sequence change on Solyc04g07292 in eight mutant lines. Specifically, (A) shows nucleotide substitutions in two target sites (two red-box indicate the modified target sequences of first and second target sites, respectively), and (B) shows amino acid substitutions (the asterisk denotes premature stop codon).

FIG. 4 relates to fruit and ovary growth of unpollinated Solyc04g07292 mutant lines. Specifically, (A) shows ovary growth in flower at 3 days after anthesis, (B) shows ovary growth according to time course (bar=1 cm), and (C) shows parthenocarpic fruit formation of 50-day-old plant (bar=5 cm).

FIG. 5 relates to parthenocarpic fruits of Solyc04g07292 mutant lines. Specifically, (A) shows parthenocarpic fruit formation (bar=1 cm), and (B) shows the percentage of parthenocarpic fruit formation (n≥100, fresh fruit weight (n >27), percentage of seeded fruit set (n=56), and number of seeds per fruit (n=12)).

FIG. 6 shows plant growth of Solyc04g07292 mutant lines (bar=5 cm).

FIG. 7 relates to parthenocarpic fruit formation under heat stress condition. Specifically, (A) shows 70-day-old plant cultivated for 30 days under heat treatment (bar=5 cm), (B) shows fruit formation of pollinated WT and unpollinated SlTPP4 mutant lines (bar=1 cm), and (C) shows the fresh weight of the fruit (n=7).

FIG. 8 relates to the phenotypes of the wild type fruit and the fruit of SlTPP4 mutant lines after auxin treatment (bar=1 cm).

### DETAILED DESCRIPTION OF THE INVENTION

Hereinafter, one or more embodiments will be described in more detail by way of examples. However, these examples are intended to illustrate one or more embodiments, and the scope of the present disclosure is not limited to these examples.

### Example 1. Plant Material and Growth Conditions

Micro-Tom, a tomato (Solanum lycopersicum L.) cultivar, was used in the present disclosure. Seeds were placed on the rockwool cube (40×40×40 mm; Grodan, Roermond, Netherlands) and cultivated with a tomato-specific nutrient solution (Daema Inc. Korea), with an electrical conductivity (EC) of 2.2 dS m-1 under a photoperiod of 16-hr light/8-hr dark at a light intensity of 150 μmol m⁻² s⁻¹ and a temperature of 25° C.

For fruit formation, one flower per flower truss was artificially pollinated by vibration, or emasculated, or 4 μl of solution containing 20 ng of 2, 4-dichlorophenoxyacetic acid (2,4-D) or 4, 000 ng of indole-3-acetic acid (IAA; Sigma-Aldrich, St. Louis, USA) and 5% ethanol and 0.1% Tween 20 was applied to the ovary of emasculated flower buds at the anthesis stage.

Six fruits per plant were maintained for the experiment. For the high-temperature experiment, the 30-day-old plants were cultivated using a plant growth chamber (VISION SCIENTIFIC, Daejeon, Korea) under the temperature and light cycle conditions of 16-hr light at 34° C. and 8-hr dark at 25° C., and a humidity of 60%.

### Example 2. Genomic DNA and RNA Extraction, and cDNA Synthesis and Amplification

Genomic DNA and total RNA were isolated using a DNeasy Plant Mini Kit (Qiagen) and a TakaRa MiniBEST Universal RNA Extraction Kit (Takara) following the manufacturers' instructions, respectively. The first-strand complementary DNA (CDNA) was synthesized from 1 ug of the total RNA using a PrimeScript II First-Strand CDNA Synthesis Kit (Takara). The synthesized CDNA was then amplified with a pair of DNAJ gene primers (forward, 5′-GAGCACACATTGAGCCTTGAC-3′ (SEQ ID NO: 13); reverse, 5′-CTTTGGTACATCGGCATTCC-3′ (SEQ ID NO: 14)) and DNA contamination was checked. Amplification was carried out in a solution containing 1 U of KOD Plus Neo polymerase, 0.2 mM deoxynucleotide triphosphates (dNTPs), a pair of specific primers (each 0.2 μM), 1.5 mM MgSO₄, and 1× buffer (Toyobo, Osaka, Japan). The thermal cycling procedure was as follows, and (ii) to (iv) were repeated for 28 cycles: (i) pre-denaturation for 5 min at 98° C., (ii) denaturation for 30 sec at 98° C., (iii) annealing for 30 sec at 60° C., (iv) extension for 1 min at 68° C., and (v) final extension for 3 min at 68° C.

### Example 3. Vector Construction and Tomato Transformation

For RNAi vector construction, the 309-bp amplicon of CAB (SEQ ID NO: 12) was obtained from the cDNA of ovaries using gene-specific primers (forward, 5′-

AGGTTTTGCGTGATAGAGGAC-3′ (SEQ ID NO: 15); reverse, 5′-CCAATTACAGTTGAGTCTTCCAAGA-3′ (SEQ ID NO: 16)). The product was cloned into a pCR8/GW/TOPO vector (Thermo Fisher Scientific, MA, USA), and then inserted into a pBI-sense, antisense gateway vector (Inplanta Innovations, Yokohama, Japan) using the Gateway LR Clonase II Enzyme mix (Thermo Fisher Scientific, MA, USA).

The vector for the CRISPR/Cas9 system, pKSE401, was used to induce mutation in Solyc04g072920. To induce knock-out mutation in Solyc04g072920, the present inventors entered Solyc04g072920 into the open access site CRISPOR (http://crispor.tefor.net/) and searched for a 20-mer highly specific sequence with the PAM sequence (NGG) to exclude off-target effects. Thereamong, the target nucleotide sequence closest to the initiation codon was selected. Thereafter, 24-mer complementary nucleotides, each having an end of 5′-aggt-3′ or 5′-ccaa-3′, were synthesized (a pair of 1936C1F, 5′-attgATAATTTCTGACCCGAGATC-3′ (SEQ ID NO: 17) and 1936C1R, 5′-aaacGATCTCGGGTCAGAAATTAT-3′ (SEQ ID NO: 18); a pair of 1936C2F, 5′-attgTCCCGGTCCACTACCGCAGC-3′ (SEQ ID NO: 19) and 1936C2R, 5′-aaacGCTGCGGTAGTGGACCGGGA-3′ (SEQ ID NO:20)).

10 μM of each pair of nucleotides was mixed and the mixture was annealed by heating up to 95° C. for 10 min and then slow cooling at room temperature. The target nucleotide sequence was ligated to pKSE401 in which an overhang end complementary to the target sequence was created using the restriction enzyme BsaI. Inserted nucleotide sequences were confirmed by sequencing with a primer set (KSEseqF, 5′-AGTCACGACGTTGTAAAACGACGG-3′ (SEQ ID NO: 21); KSEseqR, 5′-CTGCAGGCATGCAAGCTTAT-3′ (SEQ ID NO: 22)).

The transgenic tomato plant was generated by Agrobacterium-mediated transformation. The transgenic lines were employed in PCR and DNA gel blot analysis to identify homozygous-independent lines. To confirm CRISPR/Cas9-induced mutation in Solyc04g072920, gDNA of the transformant was extracted using a DNeasy Plant Mini Kit (Qiagen, Valencia, USA) and the sequence of CAB was confirmed. Three sites of leaves from each transgenic tomato were sampled, and gDNA was extracted. The gDNA of each transgenic plant was amplified using CabF, 5′-ATCGAGCGGAACATAAGTCC-3′ (SEQ ID NO: 23) and CabR, 5′-TGCAGGTTGGCAGAGTACAG-3′ (SEQ ID NO: 24) to obtain 1.1-kb PCR products which were then sequenced to confirm the mutation. Subsequently, the CabF1 and CabR primer set was also used to identify fixed mutation without transformation cassette in progeny of transgenic plants.

### Example 4. Quantitative Reverse Transcription PCR (qRT-PCR)

The relative level of mRNA was analyzed using the QuantStudio™ 6 Flex Real-Time PCR System (Thermo Fisher Scientific, Cleveland, USA) in conjunction with the TB Green Premix Ex Taq (Tli RNaseH Plus) (Takara) according to the manufacturer's instructions. The relative expression of the gene was assessed by the standard curve method with three biological replicates. Melting curve plot was analyzed to confirm the primer compatibility. SAND was used as a reference gene. The primer set used for qRT-PCR was SAND (SAND-F, 5′-TTGCTTGGAGGAACAGACG-3′ (SEQ ID NO: 25);

SAND-R, 5′-GCAAACAGAACCCCTGAATC-3′ (SEQ ID NO: 26)) and SlTPP4 (SlTPP4-F, 5′-GGTTCTGGAGATTCGTCCTAGT-3′ (SEQ ID NO: 27); SlTPP4-R, 5′-CCAAAGCCTTGTCCTCTATCAC-3′ (SEQ ID NO: 28)).

### Experimental Results

(1) Phylogenetic Analysis and the Expression of solyc04g072920

To assume the function of solyc04g072920 based on the similarities and the tissue where the gene is mainly expressed, phylogenetic analysis and the measurement of the mRNA level of solyc04g072920 in various tissues were performed. The phylogenetic analysis was carried out using the protein sequence obtained from the result of protein blast (https://blast.ncbi.nlm.nih.gov).

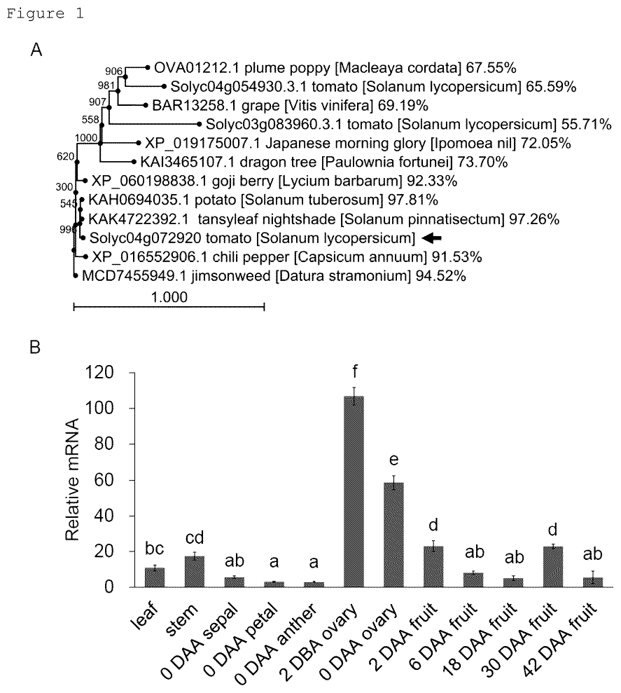

The solyc04g072920 had high identity with trehalose-6-phosphate phosphatases (TPPs) from various plant species and the gene was highly conserved between plants (FIG. 1A). The solyc04g072920 was also identified as one of 8 SITTPs in tomato and designated as SlTPP4 in a previous study (Zhang et al., 2016). The solyc04g072920 (SlTPP4) was most highly expressed in ovary at 2 days before anthesis (DBA) and the expression level decreased between 2 days and 6 days after anthesis (DAA) (FIG. 1B). The slight level of mRNA was also detected in leaf, stem, and fruit at 30 DAA. This result suggests that SlTPP4 have a role in ovary tissues.

### (2) Development of Tomato SlTPP4 Mutant Lines

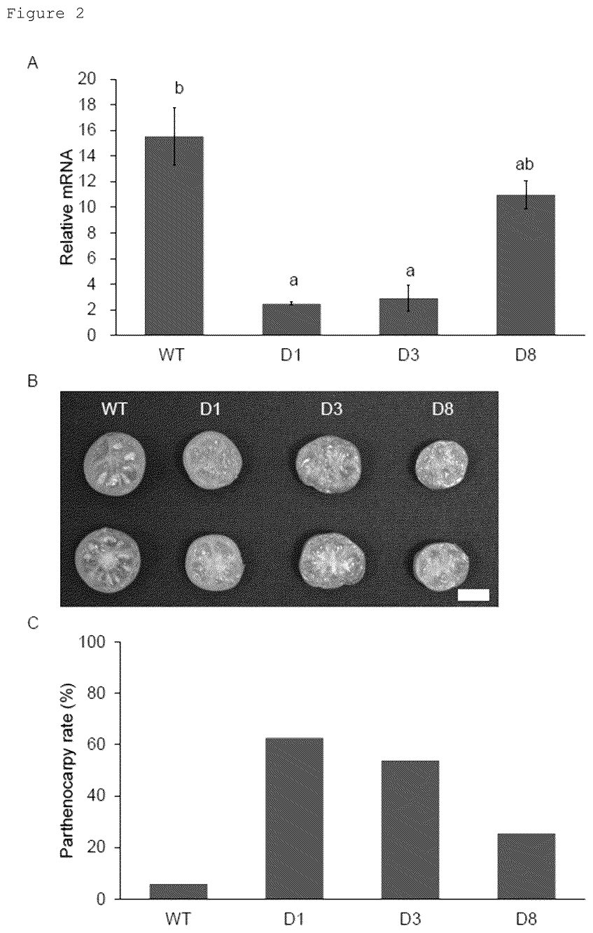

To reveal the function of SlTPP4, especially in ovary/fruit development, the present inventors obtained SlTPP4-RNA interference (RNAi) transgenic tomato lines. Surprisingly, the SlTPP4-downregulated lines showed parthenocarpic fruit formation (FIGS. 2A and 2B). In the SlTPP4-downregulated lines, the rate of parthenocarpic fruit formation exhibited a positive correlation with the downregulated level of SlTPP4 (FIG. 2C).

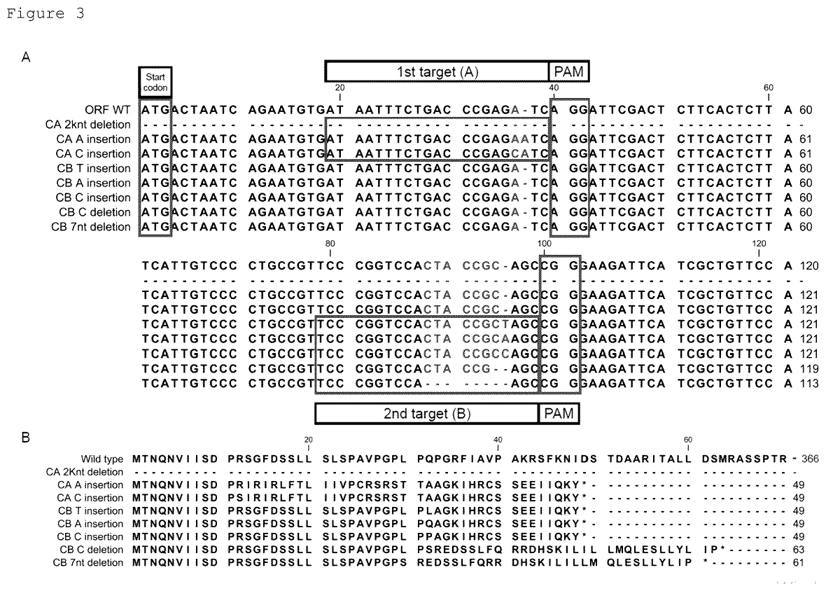

For further confirmation and for the acquirement of knockout mutants, the present inventors used CRISPR/Cas9 technology. The present inventors selected two 20-bp target sequences to induce a modification within a first exon of SlTPP4 (FIG. 3A). Finally, the present inventors obtained 3 mutants with various modified sequences in the first target site: 2,000 nucleotides deletion (CA 2Knt deletion), one adenine insertion based on the forward strand (CA A insertion), and one cytosine insertion (CA C insertion). Also, 5 mutants in the second target site were obtained: one thymine insertion (CB T insertion), one adenine insertion (CB A insertion), one cytosine insertion (CB C insertion), one cytosine deletion (CB C deletion), and 7 nucleotides deletion (CA 7nt deletion). All modified sequences in the 8 mutants caused premature stop codons with varied amino acid sequences (FIG. 3B).

### (3) Parthenocarpic Fruit of SlTPP4 Loss-of-Function Mutants

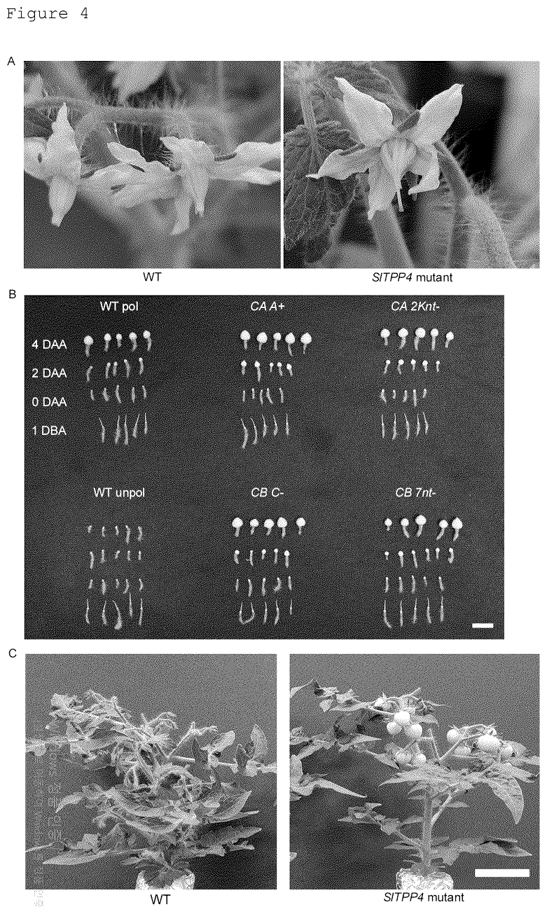

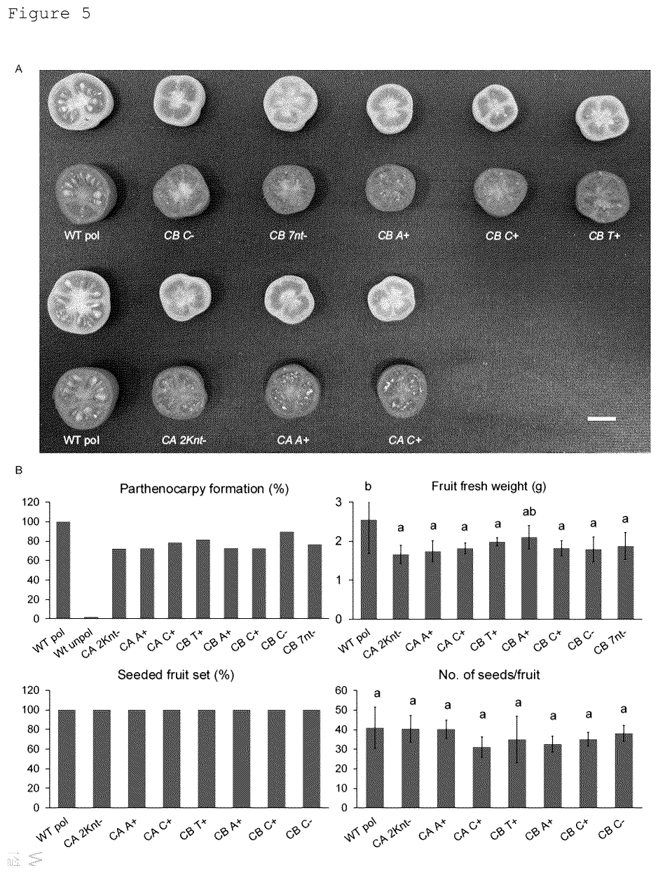

All the SlTPP4 loss-of-function (LoF) mutants showed early ovary growth in the flower stage (FIG. 4). The ovary growth in size was obviously observed in SlTPP4 LOF mutants but not in pollinated/unpollinated wild type (WT) in the anthesis stage (0 DAA), indicating that ovary development was initiated before the anthesis stage in SlTPP4 LOF mutants (FIG. 4B). In addition, the ovary size of SlTPP4 LOF mutants was generally larger than that of pollinated WT from 0 DAA to 4 DAA (FIG. 4B). Subsequently, the growing ovary successfully developed into parthenocarpic fruit (FIGS. 4C and 5A). The rate of parthenocarpic fruit formation in SlTPP4 LoF mutants was up to 90% (FIG. 5B). In addition, the fresh weight of fruits was up to 70% of that of the wild type (WT). The present inventors also investigated the ability

for seeded fruit formation. After artificial pollination by vibration, all the fruits of SlTPP4 LOF mutants contained seeds and the number of seeds per fruit was similar to that of the wild type (WT) (FIG. 5B). These results indicate that the SlTPP4 LOF mutants had the ability for facultative parthenocarpic fruit formation.

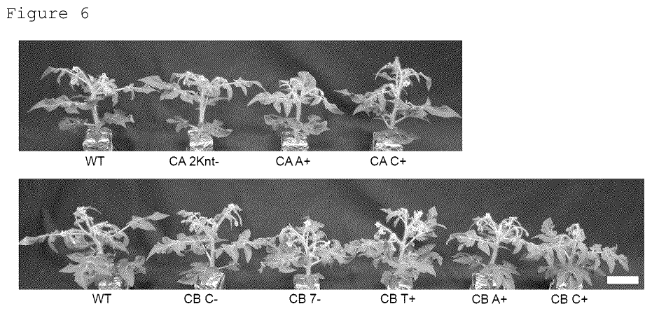

Meanwhile, no significant difference of plant growth and shape was observed between the wild type (WT) and the SlTPP4 LOF mutants (FIG. 6).

### (4) Parthenocarpic Fruit Formation Under Heat Stress Conditions

High temperature is well known as a factor, which prevents pollinated fruit setting and results in a severe reduction in tomato fruit yield. Parthenocarpic fruit formation can be an option to overcome the yield loss.

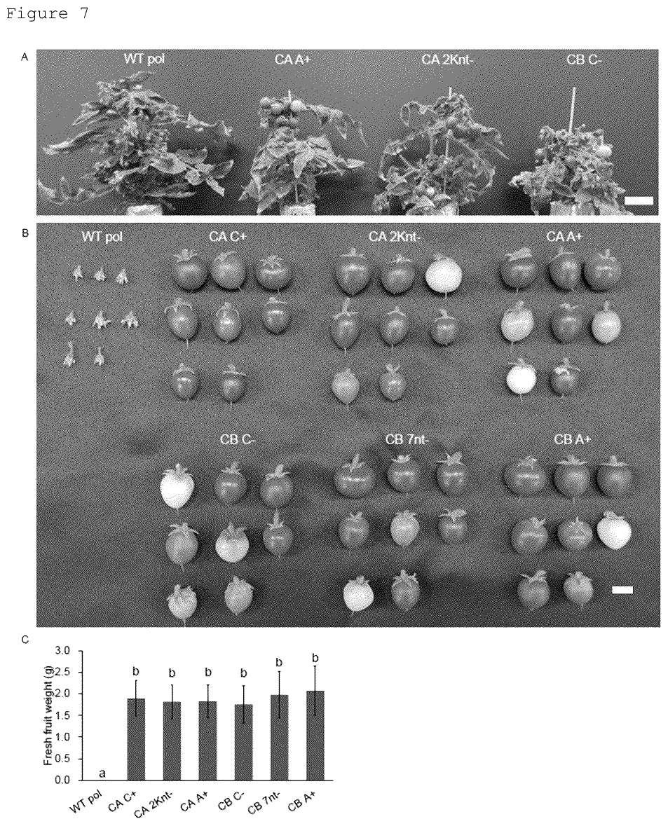

Specifically, the wild type (WT) did not form any fruit despite pollution by vibration under heat treatment at 34° C., whereas the SlTPP4 LOF mutants produced mature parthenocarpic fruits (FIG. 7). As a result of controlling the number of fruits per plant (less than 5 per plant), the average fresh weight of the fruits was up to 2 g. This result indicates that the SlTPP4 LOF mutants can produce a fruit having a fresh weight similar to that of the wild type (WT) even under heat stress (FIG. 7C).

### (5) Parthenocarpic Fruit of SlTPP4 LOF Mutants and Auxin

Previous studies reported that high indole-3-acetic acid (IAA) content (30 nmol/g of fresh weight of flower bud) or treatment with a high dose of 2,4-dichlorophenoxyacetic acid (2,4-D; >200 ng per ovary) induced the formation of parthenocarpic fruit with several protuberances (Pandolfini et al., 2002; Serrani et al., 2007). In addition, these studies also mentioned that the protuberances were not produced by pollination and GA3 treatment (Serrani et al., 2007).

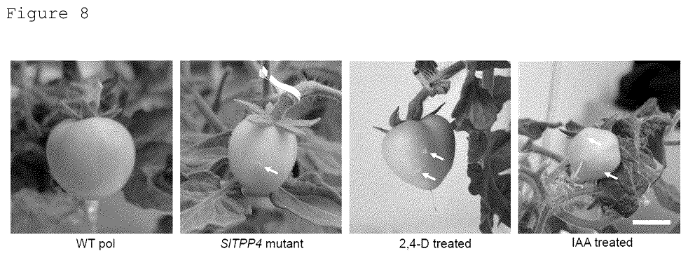

Interestingly, the parthenocarpic fruits of the SlTPP4 LoF mutants exhibited several protuberances on their fruit epidermis (FIG. 8). Treatment with 20 ng of 2,4-D and 4,000 ng of IAA per ovary also produced some protuberances on the epidermis of parthenocarpic fruits. These results suggest that parthenocarpic fruit formation is involved in auxin accumulation or relies on the auxin signaling pathway.

So far, the present disclosure has been described with reference to the preferred embodiments. Those of ordinary skill in the art to which the present disclosure pertains will appreciate that the present disclosure may be embodied in modified forms without departing from the essential characteristics of the present disclosure. Therefore, the disclosed embodiments should be considered from an illustrative point of view, not from a restrictive point of view. The scope of the present disclosure is defined by the claims rather than the foregoing description, and all differences within the scope equivalent thereto should be construed as being included in the present disclosure.

What is claimed is:

1. A composition for inducing parthenocarpy in a plant, comprising, as an active ingredient, a SlTPP4 gene expression inhibitor, a SlTPP4 protein activity inhibitor, or a SlTPP4 gene mutagen, wherein the SlTPP4 protein comprises the amino acid sequence of SEQ ID NO: 2, and the SlTPP4 gene encodes the SlTPP4 protein.

2. The composition of claim 1, wherein the SlTPP4 gene expression inhibitor is selected from the group consisting of an antisense nucleotide, small interfering RNA (siRNA), short hairpin RNA (shRNA), micro-RNA (miRNA), ribozyme, DNAyzme, and peptide nucleic acids (PNAs), which bind complementarily to a sequence of the SlTPP4 gene, a sequence complementary to the sequence of the SlTPP4 gene, or a fragment of the mRNA sequence of the SlTPP4 gene, thereby inhibiting expression of the SlTPP4 gene.

3. The composition of claim 1, wherein the SlTPP4 protein activity inhibitor is selected from the group consisting of a compound, a peptide, a peptide mimetic, a substrate analog, an aptamer, and an antibody, which bind to the SlTPP4 protein.

4. The composition of claim 1, wherein the SlTPP4 gene mutagen is either a ribonucleoprotein complex of a guide RNA specific to a target nucleotide sequence of the SlTPP4 gene and an endonuclease protein, or a recombinant vector comprising a DNA encoding a guide RNA specific to a target nucleotide sequence of the SlTPP4 gene and a nucleic acid sequence encoding an endonuclease protein.

5. The composition of claim 4, wherein the target nucleotide sequence of the SlTPP4 gene consists of the nucleotide sequence of SEQ ID NO: 3 or SEQ ID NO: 4.

6. A method for inducing parthenocarpy in a plant, comprising a step of inducing a loss-of-function mutation in a gene encoding a SlTPP4 protein comprising the amino acid sequence of SEQ ID NO: 2, or inhibiting expression of the gene, or inhibiting an activity of the SlTPP4 protein.

7. The method of claim 6, wherein the inducing of the loss-of-function mutation is achieved by introducing into a cell of the plant either a ribonucleotideprotein complex of a guide RNA specific to a target nucleotide sequence of the SlTPP4 gene and an endonuclease protein, or a recombinant vector comprising a DNA encoding a guide RNA specific to a target nucleotide sequence of the SlTPP4 gene and a nucleic acid sequence encoding an endonuclease protein.

8. The method of claim 7, wherein the target nucleotide sequence of the SlTPP4 gene consists of the nucleotide sequence of SEQ ID NO: 3 or SEQ ID NO: 4.

9. The method of claim 6, wherein the inhibiting of expression of the gene is achieved by treatment with an SlTPP4 gene expression inhibitor selected from the group consisting of an antisense nucleotide, small interfering RNA (siRNA), short hairpin RNA (shRNA), micro-RNA (miRNA), ribozyme, DNAyzme, and peptide nucleic acids (PNAs), which bind complementarily to a sequence of the SlTPP4 gene, a sequence complementary to the sequence of the SlTPP4 gene, or a fragment of the mRNA sequence of the SlTPP4 gene, thereby inhibiting expression of the SlTPP4 gene.

10. A transgenic plant or plant part exhibiting a parthenocarpic phenotype by the method of claim 6.

11. The transgenic plant or plant part of claim 10, wherein the plant contains a mutant of SlTPP4 gene represented by a nucleotide sequence selected from the group consisting of SEQ ID NO: 5 to SEQ ID NO: 11.
